(19) United States
(12) Patent Application Publication
(10) Pub. No.: US 2016/0058346 A1
(43) Pub. Date: Mar. 3, 2016

(54) DETERMINATION OF ARTERIAL CO2 PARTIAL PRESSURE

(71) Applicant: General Electric Company, Schenectady, NY (US)

(72) Inventor: Erkki Paavo Heinonen, Helsinki (FI)

(73) Assignee: General Electric Company, Schenectady, NY (US)

(21) Appl. No.: 14/474,895

(22) Filed: Sep. 2, 2014

(51) Int. Cl. A61B 5/145; A61B 5/00; A61B 5/083

(52) U.S. Cl. CPC A61B 5/14542; A61B 5/083; A61B 5/7278; A61B 5/746; A61B 5/7282

(57) ABSTRACT

A method of estimating the partial pressure of carbon dioxide in arterial blood of a patient comprises determining an arterial oxygen saturation for a patient, determining a breathing gas carbon dioxide value, and determining breathing gas oxygen value. The method further includes calculating an arterial blood carbon dioxide partial pressure for the patient based on at least the arterial oxygen saturation value, the breathing gas carbon dioxide value, and the breathing gas oxygen value for the patient.

### BACKGROUND

Monitoring carbon dioxide (CO₂) concentration in a patient is a key aspect of patient monitoring, especially monitoring in intensive care situations and/or during anesthesia. CO₂ concentrations provide information about patient acidity, which is an important descriptor of the balance between ventilation and patient metabolism. Directly determining patient arterial blood CO₂ partial pressure (PaCO₂) requires drawing a blood sample from the patient and conducting offline laboratory analysis. Such blood sampling only provides delayed information about arterial blood CO₂ concentrations, and such information is only available intermittently. For example, even for ICU patients at risk for high arterial blood CO₂ concentrations, blood samples are typically only taken once or twice daily. Furthermore, arterial sampling and the blood analysis are labor-intensive and provide opportunities for human error and sample handling.

Thus, current medical monitoring technology measures CO₂ concentrations in breathing gas expired by the patient (EtCO₂) to gauge arterial CO₂ concentrations. The international standard set for anesthesia delivery and monitoring systems mandates the use of expired CO₂ (EtCO₂) monitoring when a patient is undergoing anesthesia. The rationale is that during anesthesia the EtCO₂ is a surrogate for, or estimate of, patient arterial blood CO₂ partial pressure (PaCO₂). However, the relationship between EtCO₂ and PaCO₂ is not always direct or as expected, especially in situations involving patients with pulmonary impairments or conditions.

### SUMMARY

The present inventor has recognized that, since the relationship between EtCO₂ and PaCO₂ is not always direct or as expected, EtCO₂ is not an ideal proximate for PaCO₂ and that a better measurement of PaCO₂ is needed. In certain situations, such as in patients with a pulmonary shunt or a pulmonary emboli, the EtCO₂ can be much lower than the PaCO₂, even reaching one-third or less of the PaCO₂ value. Further, the inventor recognized that direct determination of PaCO₂ through blood sampling and lab analysis is inefficient and cannot provide the real-time data needed in manning anesthesia and intensive care environments. Accordingly, the present inventor developed the method and system described herein which reduces the difference between EtCO₂ and PaCO₂ and provides improved real-time estimation of arterial CO₂ partial pressure.

A method of estimating the partial pressure of carbon dioxide in arterial blood of a patient comprises determining an arterial oxygen saturation for a patient, determining a breathing gas carbon dioxide value, and determining breathing gas oxygen value. The method further includes calculating an arterial blood carbon dioxide partial pressure for the patient based on at least the arterial oxygen saturation value, the breathing gas carbon dioxide value, and the breathing gas oxygen value for the patient.

One embodiment of the patient monitoring system comprises a device for noninvasively measuring an arterial hemoglobin oxygen saturation value of a patient, a breathing gas analyzer, and a calculation unit. The breathing gas analyzer is configured to measure a peak carbon dioxide partial pressure in the breathing gas expired by the patient and to measure a breathing gas oxygen value. The calculation unit is configured to calculate an arterial oxygen saturation value for the patient based on the arterial hemoglobin oxygen saturation value and to calculate an arterial blood carbon dioxide indicator value of the patient based on at least the arterial oxygen saturation value and the breathing gas oxygen value for the patient.

An embodiment of the method for monitoring a patient includes estimating an amount of carbon dioxide in arterial blood of a patient by noninvasively measuring an arterial oxygen saturation value for the patient, determining a breathing gas oxygen value, measuring a peak carbon dioxide partial pressure of a breathing gas expired by the patient and calculating an arterial blood carbon dioxide partial pressure of the patient based on at least the arterial oxygen saturation value, the breathing gas carbon dioxide value, and the breathing oxygen value for the patient. The method further includes comparing the arterial blood carbon dioxide partial pressure to a peak carbon dioxide partial pressure expired by the patient and generating an alert if the arterial blood carbon dioxide partial pressure differs from the peak carbon dioxide partial pressure expired by the patient by more than a predetermined amount.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIGS. 1A-IC demonstrates oxygen and carbon dioxide partial pressure changes from breathing air to venous blood for normal lung physiology and abnormal lung physiology.

FIG. 2 provides one method of using noninvasive measurements to calculate a PaCO2 value for a patient.

FIG. 3 provides a blood hemoglobin oxygen dissociation curve.

FIG. 4 provides one embodiment of a patient monitoring system providing noninvasive estimation of the amount of carbon dioxide and arterial blood of a patient.

### DETAILED DESCRIPTION OF THE DRAWINGS

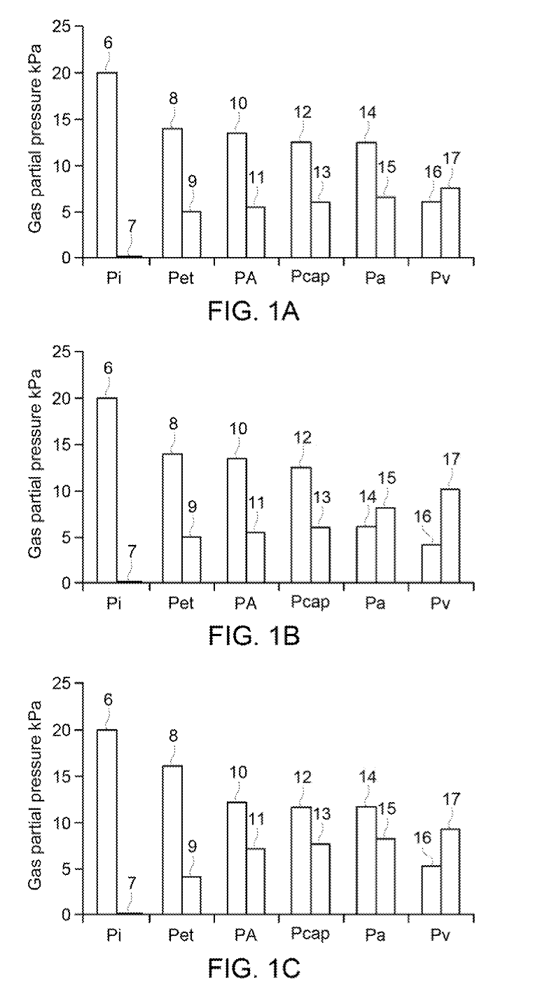

The process of ventilation, circulation, and metabolism consumes oxygen (O₂) and produces carbon dioxide (CO₂). Thus, there is a declining concentration gradient of O₂ from ambient air to venous blood and an increasing gradient for CO₂. FIG. 1 exemplifies these gradients. FIG. 1 expresses the gas partial pressure in kilopascals of O₂ and CO₂ levels during the pulmonary cycle of three different exemplary patients. FIG. 1A shows the change on O₂ and CO₂ gradients involving a normal lung, whereas FIGS. 1B and 1C demonstrate what those gradients look like for exemplary patients with lung complications.

The O₂ and CO₂ levels are depicted for six different ventilation compartments, or stages, including insufflation gas partial pressure (Pi) 6-7, exsufflation end-tidal gas pressures (Pet) 8-9, alveolar gas partial pressure (PA) 10-11, end-capillary partial pressure (Pcap) 12-13, arterial gas partial pressure (Pa) 14-15, and venous gas partial pressures (Pv) 16-17. Tissue metabolism consumes oxygen (O2) and produces carbon dioxide (CO2). Blood circulation carries the O2 to tissue and the CO2 out from the tissue. This circulation passes through lung, which comprises a large-area thin membrane between the blood and alveolar air. Gas content of the blood leaving the lung represents arterial blood while the arriving gas represents venous blood. The membrane allows gas diffusion between the capillary blood and alveolar air. Alveoli is the lung gas compartment where the breathing gas communicates with blood flow in pulmonary capillaries through a diffusion membrane allowing gas pressure equilibration between the gas and blood. Because of metabolism consuming O2 and producing CO2, the blood arriving to lungs has low O2 and high CO2 concentrations compared to the alveolar gas. Thus, in the lung the CO2 diffuses from blood to the alveoli and O2 from alveoli to blood to equilibrate the gas pressure differences. Cyclic insufflation and exsufflation (ventilation) changes the alveolar gas with ambient gas providing fresh O2 to alveoli and clearing accumulating CO2 out. Thus, the end-capillary partial pressure (Pcap) 12-13 is the capillary point where the blood is leaving the alveolar gas exchange region

As can be seen from FIG. 1, in normal lungs, the O₂ partial pressure declines from inspired gas (PiO₂ 6) to alveolar gas (PAO₂ 10), to lung capillaries (PcapO₂ 12), to arterial blood (PaO₂ 14) and finally to venous blood (PvO₂ 16). Likewise, a respective increase can be seen in the partial pressure for the CO₂ starting with the insufflation partial pressure PiCO₂ 7 (which is assumed to be 0) with incremental increases from PetCO₂ 9, to PACO₂ 11, to PcapCO₂ 13, to PaCO₂ 15, and finally to PvCO₂ 17.

FIG. 1B illustrates such gradients for a patient experiencing venous admixture, which refers to a condition where part of the blood circulation is entering the arterial system without passing through ventilated alveolar areas of the lungs causing the CO₂ of arterial blood to be higher than that of alveolar CO₂. As can be seen in FIG. 1B, the O₂ is depleted and the CO₂ levels increase as the CO₂-enriched venous blood mixes with the alveolar-equilibrated capillary blood. Thus, FIG. 1B shows an increase in PaCO₂ 15 concentration and a decrease in PaO2 14 concentration compared to FIG. 1A. Venous admixture can be caused by collapsed alveoli or occlusion of the ventilation passageway between ambient air and the alveoli.

FIG. 1C demonstrates the oxygen and CO₂ gradients for an exemplary patient experiencing alveolar dead space ventilation, which is where the insufflation gas volume does not meet the blood circulation in the alveoli and thus is experiencing gas exchange between the blood stream. The insufflation gas returns back to exsufflation, which dilutes the exsufflated concentrations toward the ambient concentrations, i.e., the O₂ concentration is increased and the CO₂ concentration is decreased as compared to normal exsufflation concentrations. Thus, PetO₂ 8 will increase and PetCO₂ 9 will decrease from the normal values. Likewise, a PaO2 decrease and increase in PaCO₂ 15 will also occur. Alveolar dead space occurs due to blockage of blood perfusion pathways, for example due to a pulmonary embolism. Finally, during long surgical operations or in intensive care under mechanical ventilation, gravitation may cause perfusion to favor certain lung areas over others, which can cause both venous admixture and alveolar dead space ventilation.

As is shown in FIGS. 1B and 1C, ventilation and perfusion mismatch may be present in patients with pulmonary impairment, increasing the difference between the expired PetCO₂ 9 and PaCO₂ 15. For example, as can be seen by comparing the PetCO₂ 9 and PaCO₂ 15 values between FIGS. 1A and 1B and IA and IC, the PaCO₂ 15 values in the patients with pulmonary impairment are much higher than the PetCO₂ values, whereas the PaCO₂ 15 value is closer to the PetCO₂ 9 value in the patient with normal pulmonary function. Thus, the value of monitoring PetCO₂ to gauge for arterial CO₂ levels is reduced. Furthermore, the importance, or health implications of a mismatch between PaCO₂ 15 and PetCO₂ 9 increases when ventilation is automatically controlled using PetCO₂ 9 as a control parameter. In such a situation, a clinician's attention toward the validity of the PetCO₂ measurement is likely reduced compared to a situation involving manual ventilation control. However, such problems can be avoided by continuous monitoring of PaCO₂ 15, which is a better indicator of the CO₂ concentrations in the patient's blood and thus of the patient's acidity.

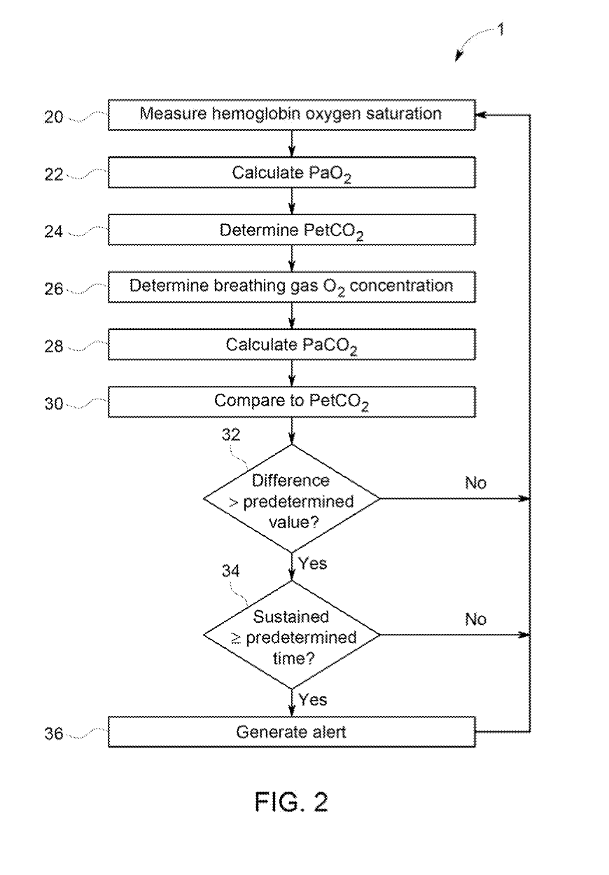

FIG. 2 exhibits one embodiment of a method for noninvasively and continuously estimating the PaCO₂ value for a patient. The hemoglobin oxygen saturation is measured at step 20, for example with a pulse oximeter. The pulse oximeter provides a noninvasive measurement of arterial blood hemoglobin oxygen saturation, and is typically taken from the periphery of a patient, such as from a fingertip or an earlobe. Such peripheral blood represents the same gas compartment as the blood leaving the lungs, i.e. PaO2. At step 22, the arterial oxygen partial pressure is calculated from the hemoglobin oxygen saturation measurement taken at step 20.

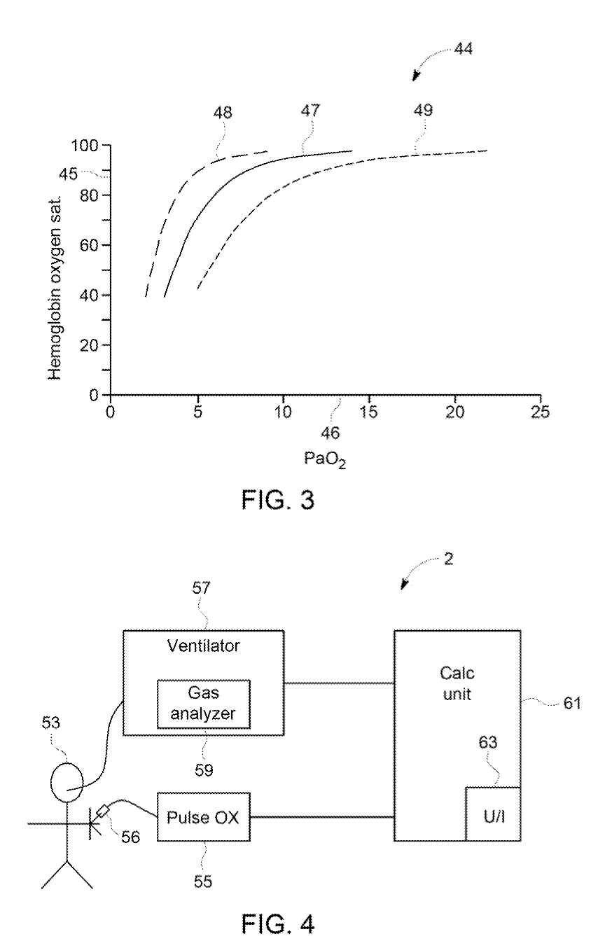

The relationship between hemoglobin oxygen saturation and PaO₂ is presented in FIG. 3. This relationship is known as the oxygen-hemoglobin dissociation curve. FIG. 3 presents the oxygen-hemoglobin dissociation curve 44, wherein the vertical coordinate 45 represents hemoglobin oxygen saturation and the horizontal coordinate 46 represents PaO₂. Line 47 describes the relationship between hemoglobin oxygen saturation and PaO₂ for a patient with a normal body pH. Line 48 is the relationship between hemoglobin oxygen saturation and PaO₂ for a patient with a high body pH, whereas line 49 describes the relationship between hemoglobin oxygen saturation and PaO₂ for a patient having a low body pH. From the appropriate oxygen hemoglobin dissociation curve 44, PaO₂ is determined from a measured hemoglobin oxygen saturation value. For example, at an oxygen saturation of 90, the corresponding PaO₂ value for a normal pH (7.4) is 7.9 kPa. On the other hand, for a patient having a pH of 7.0, which is an acidic pH (high PaCO₂), the corresponding PaO₂ value for an oxygen saturation of 90 is 12.2 kPa. For an alkaline patient having a pH of 7.8 (low PaCO₂), the corresponding PaO₂ value for an oxygen saturation of 90 is 5.0 kPa.

Returning to FIG. 2, at step 24 the PetCO₂ levels are determined for the breathing gas expired by the patient. PetCO₂ can be measured, for example, by a gas analyzer in the patient's breathing circuit and represents the maximum CO₂ value during patient exsufflation period. At step 26, the breathing gas O₂ concentration is determined. For example, a gas analyzer may be used to measure the oxygen partial pressure of the breathing gas inspired by the patient (PiO₂). Alternatively or additionally, a gas analyzer may be used to determine an end-tidal O₂ measurement or minimum O₂ partial pressure of the gas expired by the patient (PetO₂).

At step 28, PaCO₂ is calculated based on the PaO₂, PetCO₂, and breathing gas O₂ concentration values. Once the PaCO₂ value is calculated or estimated at step 28, it may be compared to the PetCO₂ value at step 30. If the difference between the PaCO₂ value and the PetCO₂ value is greater than a predetermined amount, step 32, then the patient may be experiencing high arterial CO₂ values and the clinician may need to be alerted. On the other hand, if the difference appears only in a single measurement, it could be due to measurement error or artifact. Thus, step 34 assesses whether the difference between the PaCO₂ and PetCO₂ values have been sustained for at least a predetermined amount of time. If not, then the method begins again at step 20 by measuring the hemoglobin oxygen saturation to determine whether the discrepancy between the PaCO₂ and the PetCO₂ is an error or not. If the difference between the PaCO₂ value and the PetCO₂ value has been sustained for at least a predetermined period of time, then an alert may be generated at step 36 to notify the clinician of the discrepancy between the two CO₂ values. The clinician may then take steps to verify whether the arterial CO₂ values are high and/or may adjust the patient care in order to address the high CO₂ levels. In still other embodiments, PaCO₂ calculation may be part of an automatic ventilation and/or anesthesia control algorithm. For example, an automatic ventilation control algorithm may seek to maintain a user-set, or user-defined, target PaCO₂ level.

For example, if the PaCO₂ value exceeds the PetCO₂ value by more than 2 kPa, the method may generate an alert to alert the clinician of the discrepancy. Alternatively or additionally, this method may require that the difference of at least 2 kPa be sustained for at least a predetermined period of time, such as for a set number of breath cycles or a set number of seconds, etc. In other embodiments, an alert may be generated every time the difference between PaCO₂ and PetCO₂ exceeds a predetermined value. Alternatively or additionally, the time requirement for the difference between PaCO₂ and PetCO₂ could be variable depending on the magnitude or value of the difference.

PaCO₂ values may be determined, such as at step 28 of FIG. 2, based on the difference between the breathing gas O₂ concentration and the PaO₂ value. This difference is representative of, or at least corresponds in a known way to, the difference in the PetCO₂ and the PaCO₂ values. In other words, the difference in the oxygen values is caused by the same ventilation and/or perfusion issues that would cause the difference in the CO₂ values. In one embodiment, the PaCO₂ may be calculated by adding the oxygen pressure difference between the breathing gas oxygen value and the arterial oxygen saturation value (PaO₂) to the noninvasively measured PetCO₂ value. Specifically, in one embodiment, PaCO₂ can be calculated according to the following equation:

PaCO₂=PetCO₂+k·(PxO₂−PaO₂)

wherein x designates an oxygen pressure measured from the breathing gas (e.g., either PiO₂ or PetO₂) and k is a proportionality factor. The proportionality factor k is determined empirically using clinical measurements. More specifically, the empirical determination may include collecting information of patient breathing gases and arterial oxygen saturation at the time of taking blood sample to determine true PaCO2 value. This empirical determination may also entail this collection with a large number of patients representing normal and deformed pulmonary function and covering the range of true PaCO2 values. The difference between the true PaCO₂ and estimated PaCO₂ is calculated and this calculation includes the factor k. Identifying the optimum value for the k-factor may further comprise, as an example, calculating the difference for all experimental measurements, calculating the sum of the squares of these differences, and selecting k-factor that minimize this difference. For example, if PxO₂ is PetO₂, the proportionality factor may be between 0.05 and 0.06. If PxO₂ is equal to PiO₂, then the proportionality factor may be between 0.04 and 0.05. The smaller factor for PiO₂ reflects the higher value of PiO₂ compared to PetO₂. The optimum k-factor may also be determined separately for ranges of measured values. As an example, conditions where measured breathing gas oxygen value is large or small may have different k-factors. Similarly, arterial hemoglobin oxygen saturation value low and high can have different k-factors.

The relationship between arterial and breathing gas partial pressure for oxygen and for carbon dioxide is however not constant as the equation indicates but may vary depending on level and type of lung disorders. The arterial carbon dioxide estimation accuracy can be improved by compensating with an additional difference term determined at the time of blood sampling. This term determines the difference between the true and estimated carbon dioxide partial pressures. Adding this difference to the right side of the equation calibrates the estimation to the particular lung disorder.

In another embodiment, an arterial blood carbon dioxide indicator value can be calculated in place of or in addition to the PaCO₂ value. For example, such an arterial blood carbon dioxide indicator value may be the oxygen pressure difference between the breathing gas oxygen value and the arterial oxygen saturation value (PxO₂−PaO₂). This value, which may or may not be multiplied by a proportionality factor k may be used to make a determination about or estimation of whether or not the CO₂ level in the patient's arterial blood is high. In other words, if such an arterial blood carbon dioxide indicator value, such as PxO₂−PaO₂, is greater than a predetermined amount, then it may be known that PaCO₂ is exceeding PetCO₂ by more than is desired or tolerable. Thus, such an arterial blood carbon dioxide indicator value may provide a short hand, or surrogate, for the full determination of the PaCO₂ value, and information can be gleaned about the patient's arterial CO₂ values without actually calculating the PaCO₂ and comparing it to the PetCO₂ value. For example, if the oxygen pressure difference (PxO₂−PaO₂) is high, then it may be determined that the CO₂ pressure difference (PaCO₂−PetCO₂) is also high without actually calculating that difference.

As explained above, the oxygen dissociation curve depends on body pH. Thus, the use of the oxygen-hemoglobin dissociation curve has the potential to introduce some error into the calculation. Specifically, between a normal and an acidic patient, the PaO₂ difference is 4.3 kPa, and the error resulting from this is 0.24 kPa (0.055×4.3). Such error is usually tolerable for most monitoring purposes and is much less than the potential difference between the PetCO₂ and PaCO₂ values, which may be as high as 5-7 kPa.

FIG. 4 depicts an embodiment of a different monitoring system 2 that monitors arterial blood carbon dioxide. The system 2 comprises a pulse oximeter 55 to measure hemoglobin oxygen saturation in the patient. The pulse oximeter 55 is connected to a finger probe 56 to make such measurement. The exemplary system 2 further comprises a ventilator 57 to deliver a breathing gas to a patient, which may or may not include anesthesia. The ventilator may have a gas analyzer 59 which may be configured to measure the breathing gas inspired and/or expired by the patient 53 in order to determine a breathing gas carbon dioxide value and a breathing gas oxygen value. For example, the breathing gas carbon dioxide value may be PetCO₂, whereas the breathing gas oxygen value may be PiO₂ or PetO₂, or any related gas value such as gas concentration. In other embodiments, the pulse oximeter 55 and/or the gas analyzer 59 each may be separate, stand alone devices, or they may be integrated into the ventilator or any portion of a ventilation or anesthesia providing system.

The exemplary system 2 further comprises a calculation unit 61 to calculate a value indicating carbon dioxide concentrations in arterial blood, such as the arterial blood carbon dioxide partial pressure value an/or the arterial blood carbon dioxide indicator value described above. Furthermore, the exemplary patient monitoring system 2 may comprise a user interface 63. The user interface 63 may be configured to display values to a clinician, such as PaCO₂, PetCO₂, and/or arterial blood carbon dioxide indicator values. The user interface 63 may also be configured to generate an alert to alert a clinician that carbon dioxide concentrations in a patient are too high, such as if PaCO₂ exceeds PetCO₂ by more than a predetermined amount, or an arterial blood carbon dioxide indicator value exceeds a predetermined value.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

I claim:

1. A method of estimating the partial pressure of carbon dioxide in arterial blood of a patient, the method comprising:
determining an arterial oxygen saturation value for a patient;
determining a breathing gas carbon dioxide value;
determining a breathing gas oxygen value; and
calculating an arterial blood carbon dioxide partial pressure for the patient based on at least the arterial oxygen saturation value, the breathing gas carbon dioxide value, and the breathing gas oxygen value for the patient.

2. The method of claim 1 wherein calculating the arterial blood carbon dioxide partial pressure includes determining an oxygen pressure difference between the breathing gas oxygen value and the arterial oxygen saturation value.

3. The method of claim 1 wherein determining the arterial oxygen saturation value includes measuring the arterial hemoglobin oxygen saturation for the patient and then calculating an arterial blood oxygen partial pressure.

4. The method of claim 3 wherein determining the breathing gas oxygen value includes employing a breathing gas analyzer to measure an oxygen pressure in the breathing gas inspired by the patient.

5. The method of claim 4 wherein determining the breathing gas carbon dioxide value includes employing a breathing gas analyzer to measure a peak carbon dioxide partial pressure in the breathing gas expired by the patient.

6. The method of claim 5 wherein calculating the arterial blood carbon dioxide partial pressure includes subtracting the arterial blood oxygen partial pressure from the oxygen partial pressure of the breathing gas, multiplying by a proportionality factor, and then adding the peak carbon dioxide partial pressure in the breathing gas expired by the patient.

7. The method of claim 6 wherein the proportionality factor is an empirically determined value between 0.04 and 0.05.

8. The method of claim 3 wherein determining the breathing gas oxygen includes employing a breathing gas analyzer to measure a minimum oxygen partial pressure in the breathing gas expired by the patient.

9. The method of claim 8 wherein determining the breathing gas carbon dioxide includes employing a breathing gas analyzer to measure a peak carbon dioxide partial pressure in the breathing gas expired by the patient.

10. The method of claim 9 wherein calculating the arterial blood carbon dioxide partial pressure includes subtracting the arterial blood oxygen partial pressure from the oxygen partial pressure of the breathing gas, multiplying by a proportionality factor, and then adding peak carbon dioxide partial pressure in the breathing gas expired by the patient.

11. The method of claim 10 wherein the proportionality factor is an empirically determined value between 0.05 and 0.06.

12. A patient monitoring system comprising:
a device for non-invasively measuring an arterial hemoglobin oxygen saturation value of a patient;
a breathing gas analyzer configured to:
measure a peak carbon dioxide partial pressure in the breathing gas expired by the patient; and
measure a breathing gas oxygen value; and
a calculation unit configured to:
calculate an arterial oxygen saturation value for the patient based on the arterial hemoglobin oxygen saturation value; and
calculate an arterial blood carbon dioxide indicator value of the patient based on at least the arterial oxygen saturation value and the breathing gas oxygen value for the patient.

13. The system of claim 12 wherein the breathing gas oxygen value is either a minimum oxygen partial pressure in the breathing gas expired by the patient or an oxygen pressure in the breathing gas inspired by the patient, and calculating the arterial blood carbon dioxide indicator value includes determining an oxygen pressure difference between the breathing gas oxygen value and the arterial oxygen saturation value.

14. The system of claim 13 wherein an alert is generated if the arterial blood carbon dioxide indicator value is greater than a predetermined amount.

15. The system of claim 13 wherein the breathing gas analyzer is further configured to measure a peak carbon dioxide partial pressure expired by the patient, and the calculation unit is further configured to calculate an arterial blood carbon dioxide partial pressure based on at least the arterial blood carbon dioxide indicator value.

16. The method of claim 15 wherein and the calculation unit is further configured to compare the arterial blood carbon dioxide partial pressure to the peak carbon dioxide partial pressure expired by the patient; and
generate an alert if the arterial blood carbon dioxide partial pressure varies from the peak carbon dioxide partial pressure expired by the patient by more than a predetermined amount.

17. The method of claim 12 wherein ventilation to the patient is automatically controlled base on the arterial blood carbon dioxide indicator value of the patient.

18. A method of monitoring a patient, the method comprising:
estimating an amount of carbon dioxide in arterial blood of a patient by:
non-invasively measuring an arterial oxygen saturation value for the patient;
determining a breathing gas oxygen value;
measuring a peak carbon dioxide partial pressure of a breathing gas expired by the patient; and
calculating an arterial blood carbon dioxide partial pressure of the patient based on at least the arterial oxygen saturation value, the breathing gas carbon dioxide value, and the breathing gas oxygen value for the patient;
comparing the arterial blood carbon dioxide partial pressure to a peak carbon dioxide partial pressure expired by the patient; and
generating an alert if the arterial blood carbon dioxide partial pressure differs from the peak carbon dioxide partial pressure expired by the patient by more than a predetermined amount.

19. The method of claim 18 wherein the predetermined amount is at least 2 kPa.

20. The method of claim 18 wherein the step of generating an alert if the arterial blood carbon dioxide partial pressure differs from the peak carbon dioxide partial pressure expired by the patient by more than a predetermined amount includes determining whether the difference between the arterial blood carbon dioxide partial pressure and the peak carbon dioxide partial pressure expired by the patient is sustained for at least a predetermined time period.
